(19) United States
(12) Patent Application Publication
(10) Pub. No.: US 2025/0255679 A1
(43) Pub. Date: Aug. 14, 2025

(54) PROCESSING SURGICAL DATA

(71) Applicant: CMR SURGICAL LIMITED, Cambridge, Cambridgeshire (GB)

(72) Inventors: David William Haydn Webster-Smith, Cambridge (GB); Ross Hamilton Henrywood, Cambridge (GB); Steven Michael Bishop, Cambridge (GB)

(21) Appl. No.: 18/996,029

(22) PCT Filed: Sep. 26, 2023
(86) PCT No.: PCT/GB2023/052488

§ 371 Date: Jan. 17, 2025

(30) Foreign Application Priority Data
Sep. 27, 2022 (GB) 2214067.7
Dec. 15, 2022 (GB) 2218954.2

(51) Int. Cl. A61B 34/30; A61B 90/00; G16H 30/40

(52) U.S. Cl. CPC A61B 34/30; A61B 90/361; G16H 30/40; A61B 2034/306; A61B 2090/066

(57) ABSTRACT

A surgical system comprising a processing device configured to implement a trained machine learning model, the processing device being configured to: receive first data having a first data format from a sensing device; receive additional data indicating a condition of the surgical system; in dependence on the additional data, input the first data or data derived therefrom to the trained machine learning model; and output second data having a second data format.

### FIELD OF THE INVENTION

This invention relates to data processing in surgical systems, for example to the processing of image and/or telemetry data for viewing or further analysis during robotic surgery.

### BACKGROUND

In robotic surgery, a surgeon generally controls a surgical robot from a console located at a distance from the patient. In order to view the surgical site, a camera or endoscope is commonly used and an image or video is transmitted to the console for viewing during the operation. Telemetry data can also be transmitted from sensors (for example, but not limited to, position and torque sensors) located on the surgical robot to a processing device for controlling the robot, or for data analysis.

Multispectral and hyperspectral cameras can enable the imaging of a greater number of wave bands of the electromagnetic spectrum than standard RBG cameras, which can only image in the visible light spectrum, for wavelengths of roughly 400 nm to 700 nm. These imaging techniques can reveal material properties that would otherwise not be visible with conventional cameras. This type of imaging can be beneficial in the field of surgical robotics because it can enhance contrast between structures, providing more information about the tissue. For example, it may identify blood vessels and other aspects that cannot be seen by the surgeon in the visible light spectrum. This could then allow the surgeon to make a more informed choice of where to cut, dissect and otherwise operate on tissue.

Multispectral cameras can image fewer bands, generally fewer than 10 bands from the electromagnetic spectrum, compared to hyperspectral cameras which are typically able to image more than 10 bands. The bands are discrete and can be selected from the spectrum depending on the scene to be imaged. Frequencies selected for each band could overlap with another band, or the bands selected could cover the entire continuous spectrum. Having a larger quantity of bands enables a more precise reconstruction of the continuous spectrum for each pixel. More wavebands would be able to provide more information about what is being imaged, which in turn would provide more information to the surgeon.

However, to be able to detect more wavebands the camera requires a greater number of sensors. Having more sensors can create a larger, bulkier camera at high expense. Alternatively, more bands could be included on each sensor to decrease the number of sensors needed in the camera. However, the greater the number of bands on a sensor, the worse the resolution of the image. Therefore, there is currently a trade-off between imaging a greater range of wavelengths, the resolution of the image and the size and weight of the camera.

Having a larger camera is not only a problem for multispectral or hyperspectral imaging, but is also a general problem in the field of imaging. Typically, increasing the size of the sensors in the camera and the lenses in the camera increases the number of photons captured by the sensor. Correcting lens aberrations, for example spherical aberrations or chromatic aberrations, can lead to sharper focus and less image distortion. However, this also increases the size of the camera or endoscope, as this involves adding more lenses to correct for these aberrations. Therefore, better quality images are typically taken on larger, bulkier cameras.

Furthermore, in the field of surgical robotics, a larger camera generally has a greater weight for the robot to support. Therefore, a heavier robot arm is required which, especially for a modular system, makes it less versatile and more difficult for a surgical team to set up and move around an operating theatre. A larger camera with a greater number of sensors may also have the disadvantage of greater heat dissipation, which may make the camera too hot to touch. The temperature requirement for handling the camera safely during surgery states that the temperature of the camera should not exceed 43 degrees C.

Additionally, when it is desirable to produce a better quality image, there are two factors to consider. Firstly, the ability of the camera to take high-definition images and secondly, the ability of the screen to display these images in high definition. This relies on updating both the camera and the display screen when wanting to improve the definition of videos and images to be displayed.

Finally, higher data transfer speeds are needed to transmit higher resolution images. For instance, for 4K images there are four times the number of pixels compared to 1080 p HD images, and more pixels require the transfer of more pixels per second, which increases the number of bytes the camera needs to transfer per second. Therefore, higher bandwidth requirements are generally required between the camera and video processing hardware if the resolution of an image is to be increased.

It is desirable to develop a solution for overcoming at least some of the above issues.

### SUMMARY

According to a first aspect, there is provided a surgical system comprising a processing device configured to implement a trained machine learning model, the processing device being configured to: receive first data having a first data format from a sensing device; receive additional data indicating a condition of the surgical system; in dependence on the additional data, input the first data, or data derived therefrom, to the trained machine learning model; and output second data having a second data format.

According to another aspect, there is provided a surgical system comprising a processing device being configured to: receive first data having a first data format from a sensing device; and transform the first data or data derived therefrom to output second data having a second data format. The system may be configured to receive additional data indicating a condition of the surgical system and in dependence on the additional data, transform the first data or data derived therefrom to output the second data having the second data format.

The surgical system may comprise the sensing device. The sensing device may be configured to acquire the first data having the first data format. The sensing device may be configured to acquire the first data at the first data format. Alternatively, the sensing device may be configured to acquire data having a different data format to the first data format and locally process the acquired data and output the first data having the first data format.

The processing device may be configured to, in dependence on the additional data, input the first data to the trained machine learning model; and output second data having a second data format.

The processing device may be configured to, in dependence on the additional data, input the first data or data derived from the first data to the trained machine learning model; and output second data having a second data format. For example, the processing device may apply a pre-processing model or algorithm to the first data before it is input to the trained machine learning model.

The second data format may comprise an additional aspect to the first data format.

The processing device may be configured to generate the additional aspect in dependence on the first data or the data derived therefrom using the trained machine learning model.

The trained machine learning model may be a generative model.

The first data format may represent data having a reduced quality relative to the second data format.

The second data format may have a higher spatial, frequency or temporal resolution than the first data format. The second data format may correspond to a higher level of image magnification than the first data format.

The first data format may have fewer data channels than the second data format.

The second data format may comprise a greater number of frequency bands than the first data format.

The surgical system may further comprise a robot arm having one or more joints.

The additional data indicating the condition of the surgical system may comprise data indicating a state of the robot arm. The state of the robot arm may be a spatial position of the limbs and/or joints of the robot arm.

The surgical system may further comprise a surgical instrument.

The additional data indicating the condition of the surgical system may comprise data indicating a state of the surgical instrument. The state of the instrument be a spatial position of the instrument or at least a part of the instrument and/or one or more joints of the instrument.

The surgical system may be configured to input the first data or data derived therefrom into the trained machine learning model to output the second data if the data indicating the condition of the surgical system indicates that the surgical instrument is in operation.

The sensing device may be configured to sense data relating to the position of the one or more joints or forces at the one or more joints.

The sensing device may comprise a torque sensor and/or a position sensor.

The first data format and the second data format may be respective representations of torque and/or position data.

The sensing device may be an imaging device comprising one or more image sensors.

The imaging device may be an RGB camera, a multispectral camera, a hyperspectral camera, a laser speckle camera or an endoscope.

The first data format may be a representation of an image or a video. The second data format may be a representation of an image or a video. For example, an image file or a video file (for example, having any commonly used format).

The first data may represent a complete field of view or a fraction of a field of view of the imaging device.

The first data may represent a fraction of a field of view of the imaging device. The first data may be selected in dependence on the additional data. The first data may correspond to a region of an image. The region may be a fraction of a complete image.

The second data output by the trained machine learning model may be a predicted output. The sensing device may be further configured to acquire true data having the second data format.

Where the sensing device is configured to acquire true data having the second data format, this can be used to train the machine learning model. Alternatively, the true data that the model uses to be trained may come from pre-existing datasets, which can be stored locally (stored in the system) or in the Cloud.

The system may further comprise a display device configured to display a representation of the first data having the first data format and/or the second data having the second data format.

The processing device may be configured to receive the additional data indicating the condition of the surgical system from one or more further sensing devices.

The surgical system may comprise the one or more further sensing devices. The additional data indicating the condition of the surgical system may be acquired by the one or more further sensing devices.

According to another aspect there is provided a method for data processing in a surgical system, the method comprising: receiving first data having a first data format from a sensing device; receiving additional data indicating a condition of the surgical system; in dependence on the additional data, inputting the first data or data derived therefrom to a trained machine learning model; and outputting second data having a second data format.

According to another aspect, there is provided a method for data processing in a surgical system, the method comprising: receiving first data having a first data format from a sensing device; and transforming the first data or data derived therefrom to output second data having a second data format. The method may have any of the features described above.

According to another aspect, there is provided a computer-readable storage medium have stored thereon computer readable instructions that when executed at a computer system comprising one or more processors cause the one or more processors to perform the methods above. The computer-readable storage medium may be a non-transitory computer-readable storage medium.

### BRIEF DESCRIPTION OF THE FIGURES

The present disclosure will now be described by way of example with reference to the accompanying drawings.

In the drawings:

FIG. 1 schematically illustrates an example of a surgical system.

FIG. 2 schematically illustrates an example of training a machine learning model.

FIG. 3 schematically illustrates an example of inference using a trained machine learning model.

FIG. 4 illustrates exemplary steps of a method for processing data in a surgical system in accordance with an embodiment of the present invention.

FIG. 5 schematically illustrates an image displayed on a display screen comprising a region that has been upscaled to a higher magnification.

FIG. 6 schematically illustrates an image displayed on a display screen comprising a region that has been selected and a region displaying an upscaled version of the selected region.

### DETAILED DESCRIPTION

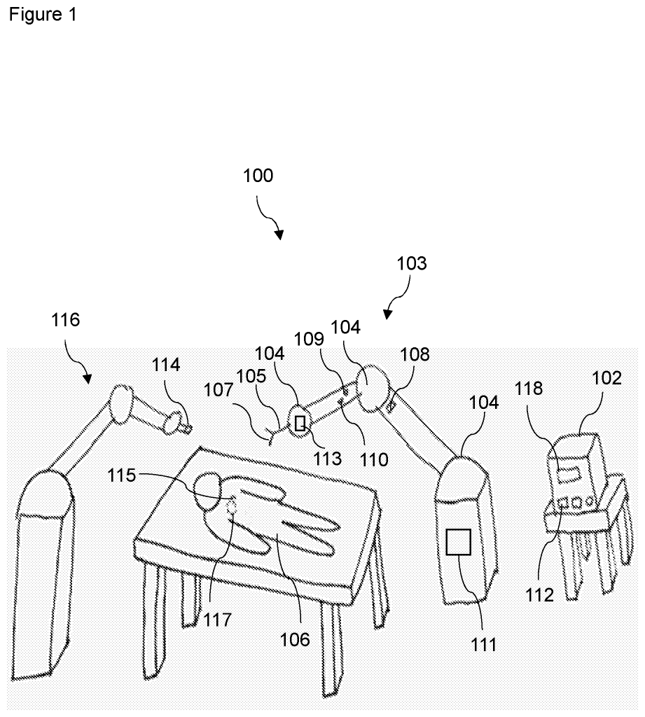

FIG. 1 shows an example of a surgical system 100. The surgical system 100 comprises a surgical robot 101 and a surgeon's console 102. The surgical robot 101 comprises one or more surgical robot arms, such as robot arm 103.

The arm 103 is articulated by means of multiple joints 104 along its length, which are used to locate a surgical instrument 105 attached to the distal end of the arm in a desired location relative to the patient 106. The surgical instrument 105 penetrates the body of the patient to access the surgical site. At its distal end, the instrument 105 comprises an end effector 107 for engaging in a medical procedure.

The joints 104 may be revolute joints, such as roll joints having an axis of rotation parallel to the longitudinal axis of a limb of the robot arm 103, or pitch or yaw joints having axes of rotation perpendicular to the longitudinal axis of a limb of the robot arm 103.

Each joint 104 of the robot arm 103 has one or more motors 108 which can be operated to cause rotational motion at the respective joint 104. Each joint 104 may have one or more position and/or torque sensors 109, 110 which can provide information regarding the current configuration and/or load at that joint. For clarity, only some of the motors and sensors are shown in FIG. 1. Controllers for the motors, torque sensors and position sensors are distributed within the arm and are connected via a communication bus to a control unit 111 of the robot arm 103.

The data acquired by the sensors 109, 110 at the joints 104 can be used to provide feedback for controlling the motors 108, or can be stored or analysed for other purposes, during or after the surgical procedure.

The control unit 111 comprises a processor and a memory. The memory stores in a non-transient way software that is executable by the processor to control the operation of the motors to cause the arm 103 to operate in the manner described herein. In particular, the software can control the processor to cause the motors (for example via distributed controllers) to drive in dependence on inputs from the sensors 109, 110 and from the surgeon's console 102. The control unit 111 is coupled to the motors 108 for driving them in accordance with outputs generated by execution of the software. The control unit 111 is coupled to the sensors 109, 110 for receiving sensed input from the sensors, and to the surgeon's console 102 for receiving input from it. The respective couplings may, for example, each be electrical or optical cables, or may be provided by a wireless connection.

The surgeon's console 102 may comprise one or more input devices 112 whereby a user can request motion of the arm in a desired way. The input devices could, for example, be manually operable mechanical input devices such as control handles or joysticks, or contactless input devices such as optical gesture sensors. The input device may alternatively be a digital input device such as a touch screen. The software stored in the memory of the control unit 111 is configured to respond to those inputs and cause the joints 104 of the arm 103 to move accordingly, in compliance with a pre-determined control strategy. The control strategy may include safety features which moderate the motion of the arm in response to command inputs. The control strategy may also adaptively optimise the trajectory of the robotic arm.

The surgical system 100 may also comprise one or more imaging devices 113, 114 to allow the surgeon to view the area of the patient 106 on which surgery is to be performed at the console 102. The imaging device may be located on the arm or may be a separate imaging device in the surgical system. The imaging device 113 is at the distal end of the robot arm 103. The imaging device 114 is at the distal end of another arm 116. Imaging devices may alternatively be located at other positions within the surgical system. For example, an imaging device 115 is located at a surgical port 117 that is installed beneath the skin of the patient. There may be multiple surgical ports installed beneath the skin of the patient, each port having an associated imaging device.

Such an imaging device may comprise one or more image sensors. The imaging device may be a camera or an endoscope. In some implementations, the camera may be a multispectral or a hyperspectral camera. The camera may be a laser speckle camera. These types of cameras can enable the imaging of more wave bands of the electromagnetic spectrum than standard RBG cameras and can reveal material properties that would otherwise not be visible and can enhance contrast between structures, providing more information about the tissue. For example, it may identify blood vessels and other anatomical structures that cannot be seen by the surgeon in the visible light spectrum. This could then allow the surgeon to make a more informed decision of where to cut, dissect and otherwise operate on tissue.

The surgeon's console 102 may also comprise a processor and a memory. The processor may process data received from the imaging device 113, 114, 115 and/or the position and torque sensors 109, 110. The memory stores in a non-transient way software that is executable by the processor to process the data, for example by implementing one or more trained machine learning model or algorithms stored in the memory.

The console 102 comprises a screen 118 or other data presentation device on which images and/or video from the imaging device(s) or telemetry data, for example from the sensors 109, 110, may be viewed by the surgeon during a surgical operation.

Thus, in summary, a surgeon at the console 102 can control the robot arm 103 to move in such a way as to perform a desired surgical procedure and view data relating to the surgical procedure. The control unit 111 and/or the surgeon's console 102 may be remote from the arm 103, 116.

It may be desirable for the sensors 109, 110 of the surgical robot and/or the imaging sensors of the imaging devices 113, 114, 115 to acquire data having a first data format that can then be processed to output data having a second data format different to the first data format. For example, one or more of the sensing devices may be configured to acquire data having a relatively low quality that is then processed to generate additional aspects from the acquired data, for example to form output data having a relatively high quality.

For example, the surgical system may comprise one or more low resolution cameras. The low resolution camera(s) may acquire data using a relatively low quality data format. Image data acquired at the relatively low quality data format may be processed as described below to output data having a relatively high quality data format. In this particular example, lower/higher quality data corresponds to lower/higher resolution data.

This may be done by inputting the acquired first data having a first data format into a trained machine learning model to transform the first data and output second data having a second data format.

In addition to implementing a trained machine learning model, the processing device of the system may be further configured to implement other algorithms for pre- and post-processing. For example, a bicubic upscaling step could be performed as a pre-processing operation, before inputting the data to the trained model. There may alternatively or additionally be some post-processing steps that are applied to the outputs of the trained model to further improve data quality data (for example, to achieve a higher resolution image or video). Therefore, the input to the model may be acquired data that has undergone one or more additional processing steps (i.e. data derived from the acquired data).

One particular category of machine learning models that may be suitable for the task of upscaling the data is generative machine learning models. Generative machine learning models are models that generate new data examples that could have plausibly come from an existing data set that is input to the model.

An example of a type of neural network that may be used for generative modelling is a Generative Adversarial Network (GAN). GANs usually work with image data and typically use convolution neural networks (CNN).

A neural network generally comprises an input layer, an output layer and one or more hidden layers and comprises a series of nodes, or neurons. Within each neuron is a set of inputs, a set of weights and a bias value. As an input enters the node, it is multiplied by the respective weight value. It may optionally add a bias before passing the data to the next layer. A weight is a parameter within a neural network that transforms input data within the network's hidden layers. A ‘weight’ of a neural network may also be referred to as a ‘parameter’ interchangeably, having the same meaning. The resulting output of a layer of the network is either observed or passed to the next layer of the network.

Weights are therefore learnable parameters within the network. A prototype version of the model may randomize the weights before training of the model initially begins. As training continues, the weights are adjusted toward the desired values to give the “correct” output.

In the training phase, the model learns its own parameters. A starting, prototype version of the model to be trained is trained using provided training data to form the trained model. The set of training data comprises input data and respective expected outputs. In the examples described herein, real data acquired using two different data formats can be used to train the model. For example, training pairs comprising data having the first data format and a true representation of the second data format can be used to train the model to produce a predicted output having the second data format. This teaches the model how to form the “correct” output.

Therefore, the processing device is configured to form the trained machine learning model in dependence on sets of training data comprising data having the first data format and respective true data having the second data format. The second data output by the trained machine learning model is a predicted output. The sensing device can be further configured to acquire true data having the second data format. The true data can be compared to the predicted output to improve the model during training.

For example, video data may be acquired at both higher and lower spatial, frequency or temporal resolution. The lower resolution data can be used as input to a prototype version of the machine learning model, which can then form a predicted output for the high-resolution data. The predicted output can then be compared with the true high-resolution data and the prototype version of the model can be updated in dependence on the accuracy of the prediction. For example, an error computed between the predicted and true data can be used to update weights of the prototype version of the machine learning model.

Another option for training is to use training data from a pre-stored or saved dataset, so that the sensors do not need to be able to obtain both low resolution and high resolution images.

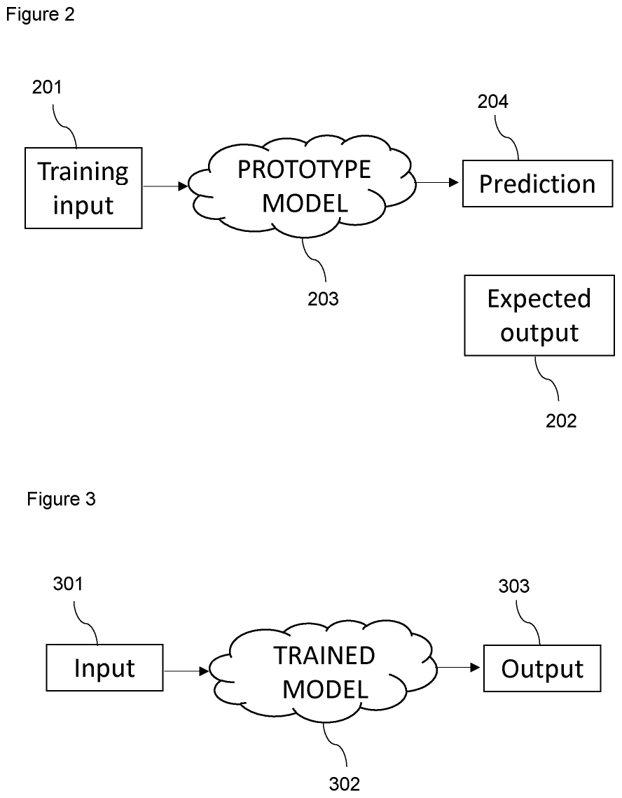

An example of the training process is schematically illustrated in FIG. 2. Training data for the model comprises pairs of data comprising an input 201 and its respective expected output 202. The expected outputs are the ‘true’ data. For example, the input 201 may comprise low-resolution data captured by a sensing device and the expected output 202 may comprise corresponding high-resolution data. The input 201 is processed by a prototype version of the model 203 and the predicted output 204 formed by the prototype version of the model 203 is compared to the expected output 202. The weights of the prototype version of the model 203 can then be updated for the next iteration of the training process to allow the model to improve its predictions and move towards convergence.

The machine learning model can therefore be trained to be able to reconstruct, for example, higher resolution image data from lower resolution image data that is input to the model.

Once training has been completed, which may be when a predetermined level of convergence of the model has been reached, the model can be output and stored for use during the inference phase. In the inference phase, the trained model is used to output one or more predictions on unseen input data.

FIG. 3 schematically illustrates an example of the inference phase using a machine learning model. Input data 301 can be an unseen input (i.e. data that has not been used to train the model, but has the same data format as the training data inputs). The input data 301 is processed by the trained model 302 to form output data 303. For example, input data 301 may be low-resolution data. The model may be trained to form a high-resolution version of the input data 301, which is output from the model at 303. The output 303 is therefore a predicted output formed in dependence on the input 301.

As mentioned above, GANs are one particular type of machine learning model that may be suitable for this task. GANs work by training two models together: the generative model and a discriminative model. The generative model generates examples from the input data. For example, in supersampling, the generative model outputs a higher resolution image from a lower resolution image input, and the discriminative model is fed with both the generated examples from the generative model and real examples (in this case, the original high-resolution images) and determines whether the generated examples are real or generated (i.e. super sampled). The discriminator model is updated based on how well it discriminates and the generator model is updated for the next iteration of the training process based on how well the output of the current generator model fools the discriminator model. In an ideal scenario, at the point of convergence of the model, the discriminator is unable to tell the difference between real and generated images every time.

To train the GAN, surgical videos, images or other data can be acquired at, for example, both higher and lower resolution. The trained model is then able to take the low-resolution data as input and fill in the missing information to generate the data in high-resolution.

Other types of models may be used, such as Convolutional Neural Networks (CNNs), which are neural network architectures that use convolutional filters to extract features in the spatial domain. For video supersampling, where the input to the model is a video comprising multiple image frames, it may be desirable to use a model architecture that considers time as a dimension, such as a recurrent neural network (RNN).

As mentioned above, the data input to the trained model may have a first data format and the output of the trained model may have a second data format, whereby one or more aspects of the second data format have been generated from the input data having the first data format.

The model may therefore be used to reconstruct information that is missing from the input data acquired by a sensing device of a surgical system and can allow aspects of the data acquired by the sensing device to be omitted and regenerated by the model during data processing. The output of the model therefore comprises one or more additional aspects compared to the input that are generated during the data processing by the model. The output of the model can be displayed to a surgeon during a surgical procedure, or may be analysed or stored for other purposes.

With the model trained using sets of training data, as described above, video or image data can be acquired at a first, relatively low resolution (for example, spatial, frequency or temporal resolution). The acquired video or image data can be input to the trained machine learning model. The model can output data having a second resolution higher than the first resolution which can then be displayed to the surgeon.

In some implementations, the trained machine learning model implemented by the processing device to process the first data may be selected in dependence on one or more of the sensing device, the first data format, the second data format, the additional aspect that is to be reconstructed during processing and additional data indicating a condition of the surgical system (as will be described in more detail later).

There may be different aspects of the image data that can be omitted from the input data to train the model to reconstruct the omitted aspect during processing. A different generative model that has been trained to reconstruct the aspect that has been omitted from the input data can be used depending on the aspect omitted from the acquired data.

The omitted aspect(s) could relate to, for example, one or more of the spatial resolution of the image or video, the frequency resolution of the video and the temporal resolution of the video. The first and second data formats may correspond to different spatial resolutions, frequency resolutions and/or temporal resolutions. First data format may have a first resolution and the second data format may have a second resolution, the second resolution being greater than the first resolution.

Spatial resolution refers to the number of pixels that make up a digital image. A high spatial resolution means that there are more pixels making up an image of a certain dimension than a low resolution image, which has fewer pixels for the same image dimension. Therefore, using a trained machine learning model taking a low resolution image as input, an image may be reconstructed that has a higher number of pixels for the same image dimension than the image captured by the camera.

Another aspect that could be reduced in the input data captured by the sensing device is the frequency resolution of an image. If some of the frequency bands are close together, one or more of the bands may be omitted from the acquired data and reconstructed by the trained model.

For example, the data may be acquired by the sensing device with particular frequency bands missing. The model may be trained using training data comprising pairs of data sampled both with and without the particular frequency bands. Using this training data, the model can be trained to reconstruct the missing aspect(s) from the input data.

In another example, the temporal aspect of a video may be reconstructed by reducing the frame rate of the acquired video and then reconstructing the omitted frames using another machine learning generative model. For example, if video at 60 frames per second is required, the model could be trained to fill in the omitted frames using video recorded at a lower frame rate, for example, of 20 frames per second.

The present approach may also be used when transferring data from the sensing device to a memory for storage or analysis. For example, high frame rate video data could be recorded and compressed. It could be uploaded at a lower frame rate to where the video data is to be stored, and then reconstructed back to the higher frame rate using a trained machine learning model.

In another example, the sensing device is a stereo endoscope camera. In this example, the stereo-endoscope camera is configured to record data on two optical channels and comprises two image sensors, one for each optical channel. The stereo-endoscope camera can capture a 3D image or video. A disparity map refers to the pixel difference between the images of these two channels, for example the right and the left channel.

Data may be acquired by the camera on only one of the two channels. Data acquisition on the second channel may not be performed. Alternatively, data may be acquired by the second channel, but may not be transmitted for processing. The data from the second channel may be reconstructed using the trained model. The disparity map could be used to train the model along with the data with one of the channels removed. The data from the missing channel could then be reconstructed by the model. Using this disparity map (or another transformation) between the present channel and the omitted channel to train the model would give a higher confidence in the model when recreating the 3D image using just one channel.

When deciding which aspect of the second data format to reconstruct from the first data, there are certain things to consider. For example, when imaging a stationary scene, the time aspect may be adjusted with confidence. For example, the frame rate of the acquired data may be reduced without losing information from the series of images, because objects in the scene are not moving. In contrast, for a moving scene, it may be more appropriate to reduce the spatial resolution of the acquired data and reconstruct that instead.

One possible way to determine the most appropriate aspect to remove from the input data and reconstruct using the trained model is to use an algorithm. The input to the algorithm may comprise one or more parameters of the scene being captured. For example, the algorithm could determine the aspect to remove from the input data based on, for example, the distance of the patient from the camera, or the distance of a particular region of interest from the camera.

For example, if a region of interest is closer to the camera than other parts of the surgical site or patient that are not of interest, then the far field image features could be removed and reconstructed, whilst acquiring the near field as normal, or vice versa if the region of interest is further from the camera than other parts of the patient or surgical site that are not of interest.

Where the data to be processed is image data (including video data), one advantage of upscaling during image processing rather than, for example, using a better camera to capture the data, is that in order to upgrade the image quality, the console screen would also need to be replaced to a high resolution capable screen, in addition to replacing the camera to one that is capable of taking better quality images. Upscaling during processing of the data means that only the screen needs to be replaced and the camera used to capture the data can remain the same. This may also mean that a smaller camera (for example, with lower resolution sensors) can be used, as the acquired images can be upscaled later. This can be advantageous in the surgical robotics field, as it is desirable for the imaging device held by a robotic arm to be as light as possible.

A further application that upscaling image data during processing can enable is an endoscope-less imaging system. Instead of having an endoscope camera that the surgeon can move and have a 3D view of the surgical field, the surgical system may instead comprise multiple small cameras. To be able to reconstruct a 3D view with multiple cameras, a point cloud of objects can be created and a virtual camera placed in that reconstructed field to determine what it would see.

This may enable the surgeon to change their view and position the virtual camera where the surgeon requires it, in a similar way to using an endoscope. Since a viewpoint is being created from a set of point cloud objects, the resolution may be poor, but the resulting image can then be upscaled during processing by inputting the image data into a trained machine learning model to achieve a good final resolution. One advantage of not having an endoscope is that one of the arms of the robot could be removed, allowing for the capability to add another instrument arm, or to have more space for the surgical team to access the surgical site.

For example, as discussed previously, the cameras or other imaging devices could be located in surgical ports 117 in the patient. Multiple low resolution cameras may be located at one or more surgical ports to reconstruct the view into one image for display to the surgeon at the console. The cameras or other imaging devices may alternatively be located in or on one or more instruments.

The above-described technique of omitting one or more aspects from the acquired data and reconstructing these aspects during processing may also be used for applications other than imaging.

The sensors 109, 110 in FIG. 1 are used to collect data at the joints 104 of the robot arm 103. Similar sensors may be used to monitor and control movement of the surgical instrument 105 and/or its end effector 107 at the distal end of the robot arm 103. The same techniques may be applied to data acquired by these sensors.

For instance, for a surgical robotic system, it is usually important to collect telemetry data. Telemetry data may include data relating to the robot's movements, torques, forces and positions per joint, which may be stored, processed and/or used as feedback to control the joints. The data processing technique described herein can be used to omit some of the telemetry data for acquisition and storage. The omitted part(s) of the acquired data can then be reconstructed for data analysis, diagnostics and other uses of the data as and when it is needed.

For example, the telemetry data may be acquired at a relatively low frequency (for example, temporal, spatial and/or frequency resolution) and then reconstructed by a trained machine learning model to form higher frequency data. This may allow the telemetry data to be transmitted to the control unit 111 of the robot arm or the surgeon's console 102 using a lower bandwidth.

Multiple trained machine learning models per joint can be used, depending on the aspect omitted from the acquired data, or different models may be used following changes in the system behaviour, or mechanical changes and/or software updates.

The acquired telemetry data relating to the movement of the robot arm, such as the joint positions and the forces and torques measured at the joints, can also be used to inform which aspects of the image data from an imaging device, such as 113, 114, 115 located on the robot arm to focus on. The telemetry data provides information about how the robot arm has moved and therefore, how the camera 113, 114 has moved. This data could also be used to train the model and therefore increase the confidence of the reconstruction. The more information that can be provided to indicate how the robot arm has moved, the more information the model has to create a more accurate reconstruction of the input data.

Additional data from the system may conveniently be used to decide whether to process the data acquired by the sensing device, or a fraction of it (for example, a fraction of the acquired data corresponding to a fraction of the complete field of view of an image sensor), by inputting it to the trained machine learning model to output data having a different data format, or whether to, for example, transmit and/or display the data to the surgeon at its acquired data format.

Such additional data may indicate a condition of the surgical system and may be acquired by one or more further sensing devices of the surgical system.

For example, a processing device may process the first data by inputting it to a trained machine learning model to output second data is if it is determined that the instrument is in a gripping, spread or cutting state. For example, if an instrument is detected to be in a gripping state, this would suggest that it is holding a needle or a piece of tissue, so at that moment the image data can be transformed by inputting the acquired first data to a trained machine learning model to output second data having, for example, a higher resolution to give the surgeon a higher quality image to assist them with performing the operation. The state of the instrument may be determined from data acquired by one or more force or position sensors of the instrument.

In another example, the system may process the first data by inputting it to a trained machine learning model in dependence on how active the surgery is. Whether surgery is active or not may be determined, for example, in dependence on a rate of change of the position of the robot arm or the instrument. The surgery may be determined to be active when the rate of change of position of the robot arm or the instrument, or the torque of a motor driving the instrument, exceeds a predefined threshold. Alternatively, the processing device may determine whether an electrosurgical tool is active and input the first data to the trained machine learning model in dependence on this determination. As a further option, the processing device may be configured to implement a smoke-removal model or algorithm on the acquired data when it has determined that an electrosurgical instrument is active, and optionally for a predetermined time period afterwards.

The acquired first data can be input to the trained machine learning model when it is determined that surgery is active. For example, in the case of image data, when it is determined that surgery is not active, the first data having the first data format as acquired by the sensing device can be displayed on the surgeon's console. When it is determined that surgery is active, the first data having the first data format can be input to a trained machine learning model to output second data having a second data format. The second data having the second data format can be displayed on the surgeon's console. In the case of position data measured by a position sensor on the robot arm or the instrument, if the instrument isn't moving then position data can be captured less frequently.

Additional data from the system may also be used to selectively decide when the sensing device is to acquire data having a first data format to be transformed using a trained machine learning model later, and when the sensing device is to acquire data directly at the second data format.

This additional data may be used to determine when to input the first data to the model. For example, it may be determined in dependence on the additional data when to input the first data to the trained model (for example, to obtain a higher resolution image) and when not to (in benefit, for example, of the temporal resolution).

For example, telemetry and/or image data may be used to infer where the distal tip(s) of the instrument is relative to the endoscope and also infer the depth of the instrument within the patient. This could be performed using a disparity map. This information could be incorporated into the model to determine areas of the image field of view that can be acquired at higher resolution and which areas can be acquired at lower resolution for upsampling using a trained machine learning model during data processing.

If it is determined that surgery is active, as described above, and the sensing device has the capability to acquire the sensed data at the second data format directly, the processing device may instruct the sensing device to acquire data using the second data format rather than the first data format. In another example, in cases where telemetry data (for example, force or position data) is measured by the sensing device, there may be specific instrument type where it may be desirable to acquire data using the second data format directly. For example, a sensing instrument that has a force sensor at the tip. In this case, the additional data may indicate the type of instrument to the processing device. The processing device may store information detailing which instrument types should acquire data directly using the second data format and which types can acquire data using the first data format that can then be input to the trained machine learning model. Therefore, in dependence on the additional data, the processing device may be configured to receive sensed data having the second data format from the sensing device.

The acquired first data (or data derived therefrom after pre-processing) may alternatively be input to the trained machine learning model in dependence on a stage of surgery being performed. For example, there may be more safety-critical stages of the operation where it is important for the surgeon to view image data a high quality, such as a suturing step.

In a further example, the first data (or data derived therefrom) may be input to the trained model in dependence on whether a virtual pivot point (VPP) is being set and/or whether the instrument is beyond the VPP. The VPP is the point in space in which the instrument pivots, typically located in the port at the incision site in the same plane as the patient's skin

In another example, the data may be input to the model in dependence on the mode that the robotic arm is in. For example, the robotic arm may be in a surgical mode when a surgical procedure is being performed. An instrument change mode allows a user to extract the instrument in a straight line from the VPP. A compliant mode allows a user to move the arm compliantly, which can be useful for example during set up of the system, for setting the VPP or to manually move the arm during surgical operation.

Depending on the mode that the robotic arm is in, either the image data or the telemetry data (or in some cases both) could be upscaled by inputting the respective data to the trained machine learning model. For example, in surgical mode, it may be desirable to upscale the image data, but it may be desirable to not perform upscaling of the image data in the compliant mode.

It may be desirable to upscale the telemetry data depending on the mode, for example by upscaling the instrument joint telemetry during surgical mode and further joints of the robotic arm during the compliant mode or instrument change mode.

Another condition of the system in which the system may process the first data (or data derived therefrom) by inputting it to a trained machine learning model to output second data having a second data format is the alarm status of the system. If a fault in the system is detected and an alarm is raised, the processing device may process and/or store data having the second data format.

Where the sensing device is an imaging device located at a surgical port, additional data relating to the location of the port relative to other parts of the patient's body may be used to selectively process the first data acquired by the imaging device using the trained machine learning model. For example, if it the port is located close to a critical vessel or organ of the patient, the image data may be processed using the trained machine learning model to output higher resolution image data.

In another embodiment, the surgeon may manually provide additional data which is used to determine whether or not to process the first data using the trained machine learning model. For example, the surgeon may make an input or a selection on the console which sends a signal to the processing device indicating, for example, the stage of the operation (such as a suturing step) in which higher quality image data is desirable. In response to the signal, the processing device may process the first data by inputting it to the trained machine learning model to output the second data. Alternatively, the system may detect the stage of the surgery or whether the instrument is in use automatically from telemetry data (for example, from force and/or position data captured by force and/or position sensors on the instrument) and transmit a signal to the processing device to indicate this. The system may alternatively extract this additional data from an image processing algorithm or a specific machine learning algorithm implemented by the processing device. Therefore, instead of the processing device receiving the additional data from one or more additional sensors of the system, the additional data could be extracted from the model itself or another model implemented by the processing device.

Using the additional data indicating a condition of the surgical system, for example whether the instrument is in operation or the current mode of the system, the system can conveniently selectively decide whether to process the image (or a part of the image) and output the image (or part of the image) at high resolution, or whether to keep the image (or a part of the image) at the originally acquired resolution.

In all of the embodiments described herein, the input to the trained machine learning model may be the data received by the processing device from the sensing device or data derived from this received data, for example by performing a pre-processing step, such as inputting the received data to a pre-processing model or algorithm to output data derived from the received data which is then input to the trained machine learning model in dependence on the additional data indicating a condition of the surgical system (for example, a part of the surgical system such as a robotic arm or an instrument).

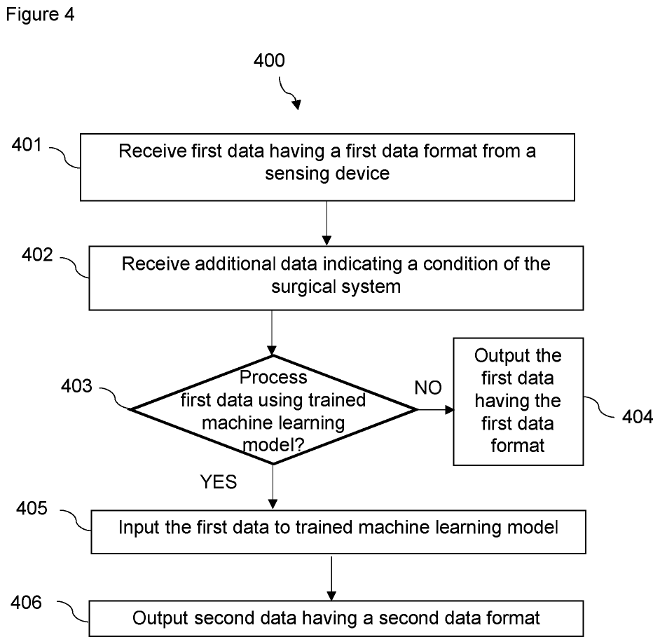

FIG. 4 shows a flowchart for an example of a method 400 for data processing in a surgical system in accordance with an embodiment of the present invention. At step 401, the method comprises receiving the first data from the sensing device. At step 402, the method comprises receiving additional data indicating a condition of the surgical system. At step 403, based on the additional data indicating a condition of the surgical system, it is decided whether to process the first data (or data derived therefrom, for example after pre-processing) by inputting it to the trained machine learning model. If it is decided that the first data should not be processed by the model, the first data having the first data format is output at step 404. The first data can then, optionally after further image processing operations (such as colour space correction or smoke removal), be displayed to the surgeon. If at step 403 it is decided that the first data should be processed, the first data is then at step 405 input to a trained machine learning model. At step 406, second data having a second data format is output. The second data can then, optionally after further image processing operations, be displayed to the surgeon.

For example, a surgical system may comprise an imaging device configured to sense first data having a first data format. For example, the imaging device may acquire image data at a relatively low resolution. A processing device is configured to receive the first data from the imaging device. The processing device also receives additional data indicating a condition of the surgical system. For example, the processing device may receive data that indicates a state of a surgical instrument of the surgical system: for example, that the surgical instrument is in use. This may be determined from data acquired by a force sensor located at the instrument indicating that the instrument is in a gripping state. In dependence on the additional data, the first data is input to a trained machine learning model which can, for example, upscale the first data having a relatively low resolution into higher resolution data that can be output and viewed by the surgeon at the console.

Embodiments of the present invention may, for example, provide improved vision capabilities to the surgeon, enabling the use of a smaller and lighter camera, as well as lower bandwidth rates for efficient data transfer.

One particular advantage of this is that low-resolution videos are able to be played in high-resolution by upscaling the video during processing into much higher definition video, for example to enable a standard definition TV film to be displayed on a 4K television. This means that low-definition videos do not need to be recorded on high-definition cameras with expensive optics, but can still be watched in high definition on high-definition screens located at the surgeon's console.

Upscaling the resolution of the image in processing can also enable the ability to obtain an image that uses the information acquired in several different frequency wavebands therefore increasing the detail and information presented to the surgeon. This can conveniently enable the use of a camera with a greater number of bands per sensor, enabling a smaller and lighter camera without compromising on the resolution of the final displayed image.

Another advantage, specifically for a surgical robotic system, is the added insight of where the surgeon is likely to be focussing on. This is likely to be where the instruments are positioned. Therefore, telemetry data from the joints of a robot arm and/or from the instrument can also be used to enable the determination of a spatial region of the image data to process and a spatial region (or regions) not to process.

This may enable a higher level of confidence for the image data in the region that the surgeon is most focussed on. For example, the periphery of the image could be either captured at a lower resolution, or removed completely for the transmission, and reconstructed later, as the surgeon may be less focussed on this region compared to the central region of the image.

This way, there may be more certainty at the specific regions of interest. This may be useful for storing the video or transferring the video to different screens or locations, as it enables a reduction in the bandwidth. Therefore, a partial image comprising the more important areas of the image may be kept and transmitted and the entire image may be reconstructed from the partial image once it has been sent to the desired location.

This can also allow the system to store the received data from the sensing device, occupying less memory of the system, since it will be at a lower resolution. Once it is stored after acquisition in real-time at lower resolution, it can be decided whether to upscale the data during or after the surgery and display it at a higher resolution after upscaling.

As described above, the system described herein may be configured to apply an upscaling function to first data or data derived therefrom. The first data or data derived therefrom may correspond to one or more regions of an image, or a whole image. The first data or data derived therefrom may therefore correspond to at least part of an image. The first data may alternatively correspond to other types of data, such as position or force telemetry data. For example, a trained machine learning model (or other method such as bipolar interpolation) can be applied to the first data or data derived therefrom from those one or more regions of the image. The upscaling function can transform the first data corresponding to the one or more regions of an image to output second data for the one or more regions. The second data may have a higher resolution (for example to 4K), frame rate or other parameter than the first data. This can allow more detail to be displayed for a specific region of interest in the image (for example, more detail of small vessels, nerves, etc). The upscaled region(s) of the image therefore have a second data format, relative to the non-upscaled region(s) of the image, which have a first data format.

Thus, as used herein ‘upscaling’ means transforming first data or data derived therefrom, the first data having a first data format, to output second data having a second data format.

In some examples, by upscaling only a specific region of interest of the complete image, the computational cost can be reduced compared to upscaling the whole image. Furthermore, users may find it overwhelming or unnecessary to have, for example, high resolution detail of all areas of the image and may prefer to only have the higher detail applied to the region of the field of view on which they are focusing and/or operating on. However, in other applications it may be desirable to upscale the whole image.

The system may be configured to operate according to different data processing modes. The system may be configured to operate according to an upscaling data processing mode whereby at least some of the first data having a first data format (such as image data, from part or all of an image, or telemetry data) or data derived therefrom is transformed to output second data having a second data format. The upscaling mode differs from the standard data processing mode of the system, whereby in the standard mode none of the first data or data derived therefrom is transformed, using a trained machine learning model or other method, to output second data as described herein.

In some embodiments, the system may be controllable to allow the upscaling mode to be switched on and off (i.e. to enable and disable the upscaling mode) as desired by the user. Alternatively, the system may be configured to automatically enable and/or disable the upscaling mode. When the upscaling mode is disabled, the complete image may be displayed using standard parameters (such as a standard predefined resolution, etc). When the upscaling mode is enabled, all or part of the image may be upscaled accordingly, according to any of the embodiments described herein.

The enabling and/or disabling of the upscaling mode can be performed, for example, from one or more input devices 112 of the console, for example using a hand controller. This may be performed via a selection on a menu of a display screen on the surgeon's console, which may be selected via a button of the input device. The functions of the button of the input device could be dynamic, meaning that they can change depending on the stage of the surgical operation the surgeon is performing.

The enablement and/or disablement of the upscaling mode may alternatively be voice activated, activated in response to gesture detection (for example by detecting a sequence of movements from the surgeon), by head tracking, eye tracking, or via a display panel that can receive user inputs.

The enablement and/or disablement of the upscaling model may also be semi-automatic. For example, a display can show the different surgical steps that the surgeon will perform in the current surgical procedure, and new system functionalities may be automatically enabled in each specific step (including the upscaling mode for one or more steps). Therefore, the upscaling mode may be enabled and/or disabled in dependence on the step of the current surgical procedure being performed.

In another example, the upscaling mode may be enabled and/or disabled in dependence on the amount of memory available at one or more memories of the system. For example, the upscaling mode may be disabled if the amount of memory available in the system is less than a predetermined threshold.

The enablement and/or disablement of the upscaling mode may be performed in dependence on one or more features detected from an image. The image may depict a surgical scene. For example, if it is determined from an image captured by an image sensor that the instrument is outside the patient (for example, from one or more features extracted from an image, which may be used to classify the image or contents thereof), the upscaling mode may be disabled.

The enablement and/or disablement of the upscaling mode may be performed automatically, for example using a timer. The upscaling mode may be disabled after a predetermined amount of time has elapsed since the upscaling mode was last enabled. If a user desires to extend the duration of the upscaling mode, the user can manually enable the upscaling mode again at the end of the predetermined period of time. This may ensure that the user does not forget to disable the upscaling mode when it is no longer desired, which may help to reduce computational costs. Alternatively, if a certain condition of the system is met in which the upscaling is to be applied when the predetermined time period has elapsed, the timer may be automatically reset to extend the enablement of the upscaling mode for a further predetermined time period.

The upscaling mode may also feature tuneable settings and/or parameters. The settings and/or parameters may be chosen by a user of the system. For example, the system may be configured to receive input from a user indicating a desired level of upscaling (for example, fixed integers such as ×2, ×4 resolution, magnification etc, or a level on a continuous scale) to be applied to the first data or data derived therefrom.

The system may take into account that an upscaling mode transforming the first data or data derived therefrom to, for example, second data having a higher resolution, may be computationally slower than using the standard mode or an upscaling mode with a lower resolution. Therefore, for image data, the system may be configured to display a still or slowed-down (for example, a reduced number of frames per second of a video) version of the second data. For example, where the image data is upscaled to a high resolution, the user can benefit from the greater level of detail of anatomical structures for a specific period of time.

In addition to the methods described above, the region of an image to be upscaled (such that the first data or data derived therefrom corresponding to that image is transformed to output second data) may be further determined as follows.

The region of the image may be determined by detecting the location of the distal tip of a surgical instrument in the image, and the region may be that location (for example the pixels that include a part of the instrument tip in the image) and, optionally, additionally a predetermined number of pixels around the location.

The region may be determined in dependence on information contained in the image. For example, using image analysis information (using image characteristics and/or recognition and segmentation of anatomical structures) or using kinematic information from the robotic arm and instrument (for example from position and/or torque sensors, as described above). For example, the region to be upscaled may be determined by detecting a region of the image that contains one or more anatomical structures, such as one or more vessels or organs or parts thereof, which may be predetermined.

The methods used to determine the region of the image may be dependent on the surgical instrument being used. For example, it may not be suitable to predict the position of the instrument using time interpolation with a sharp instrument, for example comprising a blade, but for a more blunt instrument, such as a grasper, this may be acceptable.

Optionally, more than one user may select the region. Users of the system may comprise the surgeon and one or more members of a surgical team. For example, in addition to the surgeon being able to select the region of the image to upscale, one or more members of the surgical team may select the region from auxiliary display screens (in addition to the display screen of the surgeon's console) in the operating room.

The system may also be configured so that a user can select where to display the upscaled image (corresponding to the second data) or the region of the image that has been upscaled. For example, the user may command the system to take a snapshot of the upscaled image while in the upscaling mode and display that image for a period of time on a display screen (which may be a touchscreen display). This display screen may be an additional display screen to the display screen of the surgeon's console. This way, the computation occurs over a short period of time, but the upscaled image can be viewed for a longer period of time.

In another implementation, a machine learning model may be trained to learn when during a particular surgical procedure a user normally uses the upscaling mode. The model may be trained based on previous data obtained during one or more surgical procedures, which may be for a particular surgeon. Once trained, the model may suggest use of the upscaling mode at particular points in a surgical procedure as determined by the model (which a user may confirm, causing the system to enable the upscaling mode), or based on the output of the model, the system may automatically transition to the upscaling mode at particular stages of a future surgical procedure of that particular type. The model could also be trained to identify one or more anatomical structures, and cause the system to upscale one or more regions of the image determined as containing those structures (for instance, high vasculature tissues).

The upscaling of the image or a region of the image (i.e. the transformation of the first data or data derived therefrom to second data) may be performed in real time or retrospectively. Therefore, the current frame of the display or any previously recorded frames of the display can be upscaled. This may be particularly useful in an event where a surgeon wants to see how the anatomical structure appeared at the beginning of the surgery, to compare it to how it appears after the surgical steps they have performed.

In other embodiments, regions of an image that have each been transformed using different levels of upscaling (or no upscaling) may be displayed on the same display screen. This can allow for variable zoom across the screen. For example, a user may select a region of the image using an icon on the screen such as a crosshair and the selected region may be upscaled to a higher resolution.

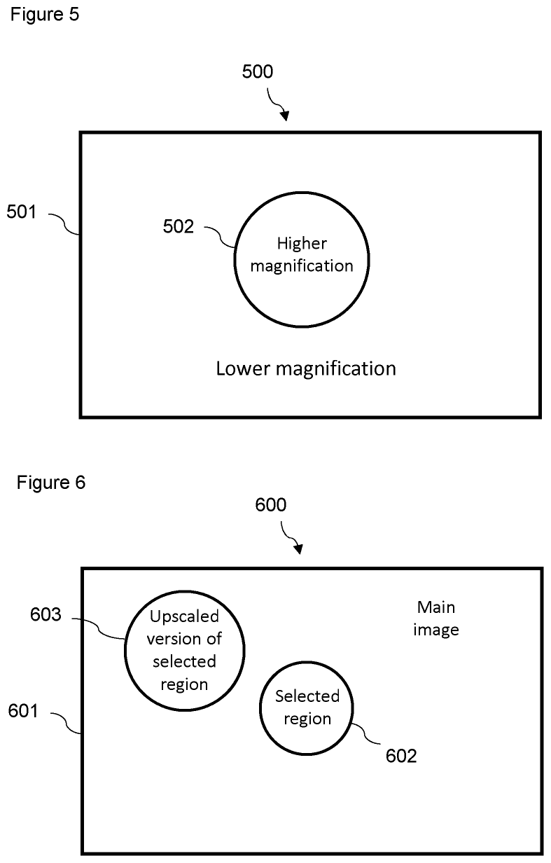

As schematically illustrated in the example of FIG. 5, a main image 501 may be displayed on a display screen 500. A region 502 of an image has been zoomed in to display the region at a higher magnification compared to the rest of the image. The region 502 can also be upscaled to a higher resolution. When a region of the image is magnified, the region may have a relatively low resolution (and may appear pixelated). An upscaling function may be applied to the region of the image that has been magnified to transform the region of the image to a higher resolution to compensate. This may improve the sharpness of edges and the amount of detail visible in the region of the image. In FIG. 5, the region has been selected and displayed on the display screen 500 at the centre of the main image 501. There is therefore a region of higher magnification and resolution corresponding to the region selected for upscaling, and regions of relatively lower resolution and magnification in the other areas of the image, for example in the peripheries of the image 501. The transition between the two regions could be blunt or a graded transition. The shape of the region 502 may have any arbitrary shape, but is shown as being circular in the example of FIG. 5.

The system may be configured to cause the region to be displayed at an area of a display screen. The display screen may be a display screen of the console or another display screen of the operating theatre. This area may be a fraction of the total area of the display screen. This area may be movable within the total area of the display screen. The system may be configured to move the area in response to a user input.

In some embodiments, a zone of interest may be defined (for example by an outline such as a circle) to select the region of the image to be upscaled and the upscaled image of this region may be placed elsewhere in the image. This can enable the selected region and the upscaled region to have different sizes and can ensure that parts of the image are not permanently covered by the upscaled region if located over the main image and therefore lost.

As schematically illustrated in FIG. 6, a main image 601 may be displayed on a display screen 600. The region 602 is the region of the main image selected or determined for upscaling. The region 603 contains the upscaled version of the selected or determined region 602.

The user may be able to move the region 603 around the display screen 600 and adjust the size and shape of the region 603. The user may be able to select the region 603 and move it on screen (for example by clicking and dragging the region 603, which may be performed using the hand controller(s) or other input device of the console). Alternatively, the user may be able to select the position of the region 603 on the screen 600 from a set series of options.

Therefore, the selected/determined region to be upscaled 602 and the displayed representation of the upscaled region 603 may be variable in size and/or position within the main image 601 on the same display screen 600, or on a separate display screen. The position and/or size of the region within the main image may be controllable in response to a user input.

The region may be moveable in dependence on the region of the image (for example, so as not to cover another area of the image containing a feature of interest), or based on user input or eye tracking. If a region of the image is upscaled compared to the rest of the image, there could additionally be some digital image stabilisation processing performed for the region of the image. This could compensate for effects such as camera wobble, patient respiration, etc. Therefore, the second data corresponding to the region may undergo further processing relative to the first data or data derived therefrom that is display for the rest of the image.

In another example, a user may define a free-form region of the image to be upscaled, for example by using a tablet and stylus, a hand controller input device or instrument tip to select the region to be upscaled. This may be used to obtain, for example, increased magnification, resolution or frame rate in that region.

The selection of the region to be upscaled from the main image view may be performed in combination with the enable/disable selection described above via a combined gesture or other indicator.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A surgical system comprising a processing device configured to implement a trained machine learning model, the processing device being configured to:
receive first data having a first data format from a sensing device;
receive additional data indicating a condition of the surgical system;
in dependence on the additional data, input the first data or data derived therefrom to the trained machine learning model; and
output second data having a second data format.

2. The surgical system as claimed in claim 1, wherein the second data format comprises an additional aspect to the first data format.

3. The surgical system as claimed in claim 2, wherein the processing device is configured to generate the additional aspect in dependence on the first data using the trained machine learning model.

4. The surgical system as claimed in claim 3, wherein the trained machine learning model is a generative model.

5. The surgical system as claimed in claim 1, wherein the first data format represents data having a reduced quality relative to the second data format.

6. The surgical system as claimed in claim 1, where the second data format has a higher spatial, frequency or temporal resolution than the first data format.

7. The surgical system as claimed in claim 1, wherein first data format has fewer data channels than the second data format.

8. The surgical system as claimed in claim 1, wherein the second data format comprises a greater number of frequency bands than the first data format.

9. The surgical system as claimed in claim 1, wherein the surgical system further comprises a robot arm having one or more joints.

10. The surgical system as claimed in claim 9, wherein the additional data indicating the condition of the surgical system comprises data indicating a state of the robot arm.

11. The surgical system as claimed in claim 1, wherein the surgical system further comprises a surgical instrument.

12. The surgical system as claimed in claim 11, wherein the additional data indicating the condition of the surgical system comprises data indicating a state of the surgical instrument.

13. The surgical system as claimed in claim 12, wherein the surgical system is configured to input the first data into the trained machine learning model to output the second data if the data indicating the condition of the surgical system indicates that the surgical instrument is in operation.

14. The surgical system as claimed in claim 9, wherein the sensing device is configured to sense data relating to the position of the one or more joints or forces at the one or more joints.

15. The surgical system as claimed in claim 14, wherein the sensing device comprises a torque sensor and/or a position sensor.

16. (canceled)

17. The surgical system as claimed in claim 1, wherein the sensing device is an imaging device comprising one or more image sensors.

18. (canceled)

19. The surgical system as claimed in claim 17, wherein the first data format and the second data format are respective representations of an image or a video.

20. The surgical system as claimed in 17, wherein the first data represents a complete field of view or a fraction of a field of view of the imaging device.

21. (canceled)

22. The surgical system as claimed in claim 1, wherein the second data output by the trained machine learning model is a predicted output and wherein the sensing device is further configured to acquire true data having the second data format.

23.-24. (canceled)

25. A method for data processing in a surgical system, the method comprising:
receiving first data having a first data format from a sensing device;
receiving additional data indicating a condition of the surgical system;
in dependence on the additional data, inputting the first data or data derived therefrom to a trained machine learning model; and
outputting second data having a second data format.
